(19) United States
(12) Patent Application Publication
(10) Pub. No.: US 2010/0104565 A1
(43) Pub. Date: Apr. 29, 2010

(54) COMBINATIONS COMPRISING DMXAA FOR THE TREATMENT OF CANCER

(75) Inventors: Colin Green, London (GB); Lloyd Kelland, London (GB); Sally Kelland, Reigate (GB); Gail Rowlinson-Brusza, London (GB)

Correspondence Address: FOLEY & LARDNER LLP, 975 PAGE MILL ROAD, PALO ALTO, CA, 94304, US

(73) Assignee: ANTISOMA RESEARCH LIMITED

(21) Appl. No.: 12/064,632

(22) PCT Filed: Aug. 25, 2006
(86) PCT No.: PCT/GB2006/003207

§ 371 Date: Sep. 8, 2009

(30) Foreign Application Priority Data
Aug. 26, 2005 (GB) 0517387.7

(51) Int. Cl. A61K 39/395; A61K 31/352; A61P 35/00; A61K 31/517; A61K 31/5377

(52) U.S. Cl. 424/133.1; 514/455; 424/145.1; 514/266.4; 514/234.5

(57) ABSTRACT

The present invention relates to combinations of the xanthenone acetic acids class such as 5,6-dimethylxanthenone-4-acetic acid (DMXAA) and EGFR signalling pathway inhibitors. More particularly, the invention is concerned with the use of such combinations in the treatment of cancer and pharmaceutical compositions containing such combinations.

The present invention relates to combinations of compounds of the class having the formula (I) as defined below, for example compounds of the xanthenone acetic acid class having the formula (II) as defined below, such as 5,6-dimethylxanthenone-4-acetic acid (DMXAA), or a pharmaceutically acceptable salt, ester or prodrug thereof and EGFR signalling pathway inhibitors. For example, the present invention relates to synergistic combinations of compounds of the class having the formula (I) as defined below, for example compounds of the xanthenone acetic acid class having the formula (II) as defined below, such as 5,6-dimethylxanthenone-4-acetic acid (DMXAA), or a pharmaceutically acceptable salt, ester or prodrug thereof and EGFR signalling pathway inhibitors. More particularly, the invention is concerned with the use of such combinations in the treatment of cancer. The present invention also relates to pharmaceutical compositions containing such combinations.

5,6-dimethylxanthenone-4-acetic acid (DMXAA) is represented by the following formula:

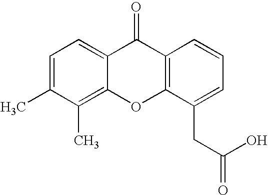

Three phase I clinical trials of DMXAA as a monotherapy have recently been completed, with dynamic MRI showing that it induces a significant reduction in tumour blood flow at well-tolerated doses. DMXAA is thus one of the first vascular disrupting agents (VDAs) for which activity (irreversible inhibition of tumour blood flow) has been documented in human tumours. These findings are in agreement with preclinical studies using syngeneic murine tumours or human tumour xenografts, which showed that its antivascular activity produced prolonged inhibition of tumour blood flow leading to extensive regions of haemorrhagic necrosis.

However, in these phase I clinical trials of DMXAA there were very few tumour responses, demonstrating that DMXAA alone does not have significant potential in cancer treatment as a single agent. Therefore, there is a need to identify compounds that could have a synergistic effect with DMXAA.

There is a new class of cancer drugs available that are not cytotoxics, but block the epidermal growth factor signalling pathways. Examples include Erbitux™ (cetuximab), a monoclonal antibody binding to epidermal growth factor receptor (EGFR) and Tarceva™ (erlotinib) and Iressa™ (gefitinib), small molecules that inhibit cell signalling in the EGFR pathway. We have surprisingly found that DMXAA may act synergistically with these new agents, enhancing their anti-cancer activity.

### EGFR Signalling Pathway Inhibitors

Tumours have been found to overexpress certain growth factors that enable them to proliferate rapidly, one of which is EGF. Activation of EGFR by binding of EGF and formation of an active receptor dimer induces phosphorylation of the tyrosine kinase in the intracellular domain of the receptor. The ras protein initiates a cascade of phosphorylations which result in activation of mitogen activated protein kinase (MAPK). MAPK triggers events in the nucleus that result in cell division. As a result, overexpression of EGF, or of EGFR on the cell surface can result in uncontrolled cell division characteristic of cancer. Expression levels of EGF and EGFR are negatively correlated with prognosis and survival in cancer, and inhibiting the signalling pathway has been shown to improve survival.

The EGFR pathway is targeted by Erbitux™ (cetuximab, a chimeric monoclonal antibody marketed for colorectal cancer by Imclone and Bristol-Myers Squibb in the US and Schering in Europe), which binds to EGF receptors, blocking EGF from binding to them. Tarceva™ (erlotinib, marketed by Genentech and OSI Pharmaceuticals in the US and Roche elsewhere) and Iressa™ (gefitinib, marketed by AstraZeneca), small molecules marketed for non-small cell lung cancer, inhibit phosphorylation of the intracellular tyrosine kinase, interfering with cell signalling. This limits the uncontrolled cell division caused by overstimulation of the EGFR signalling pathway.

Of the EGFR signalling pathway inhibitors, only Tarceva™ has demonstrated a survival advantage in phase III trials, with both Erbitux™ and Iressa™ being approved based on tumour response rates. Since its approval Iressa™ has completed a number of phase III trials, which found that it did not extend median survival, despite the improvement in response rate over standard care.

### Previous EGFR Signalling Pathway Inhibitor Combination Studies

Clinical trials of the EGFR signalling pathway inhibitors do not suggest that they are likely to show synergy with vascular targeting anti-cancer agents. Erbitux™ is approved for use as a monotherapy or in combination with irinotecan, a non-vascular targeting cytotoxic.

Both Iressa™ and Tarceva™ have been tested with combinations that include paclitaxel, a compound known to have anti-angiogenic properties secondary to its cytotoxic activity, with no evidence of benefit. For both products, two trials failed to show a benefit of adding the EGFR signalling inhibitor to standard chemotherapy. Iressa™ is indicated only as a monotherapy because two large, controlled, randomised trials showed it to give no survival benefit when used first-line in combination with chemotherapy that included a platin and another agent, which could be paclitaxel. Tarceva™ has been similarly unsuccessful in demonstrating a survival benefit when combined with carboplatin/paclitaxel or cisplatin/gemcitabine. Tarceva™ has demonstrated a survival benefit in pancreatic cancer patients when combined with gemcitabine, a non-vascular targeting cytotoxic cancer drug.

### Previous DMXAA Combination Studies

DMXAA has previously been demonstrated to have synergy with a number of agents in xenograft studies. These agents include widely used cytotoxic chemotherapies such as taxanes (paclitaxel and docetaxel), platins (cisplatin and carboplatin), vinca alkaloids (vincristine), antimetabolites (gemcitabine), topoisomerase II inhibitors (etoposide) and anthracyclines (doxorubicin). It is believed that the synergy arises because DMXAA causes necrosis in the centre of tumours, but seems to leave a viable rim of cancer cells. These are targeted by the cytotoxic agents which primarily act on rapidly proliferating cells. None of these chemotherapy agents are known to affect the EGFR signalling pathway.

DMXAA is currently in two phase II trials examining its anti-tumour efficacy in combination with paclitaxel and carboplatin, and one trial combining it with docetaxel. The cytotoxic effect of the taxanes is caused by interference with tubulin, which prevents normal mitosis (cell division). A secondary effect is disruption of newly formed blood vessels, since the cells of the new vascular endothelium depend on tubulin to maintain their shape. However, the cytotoxic effect is overriding at higher doses, such as those used in chemotherapy. Any synergy between DMXAA and the taxanes is thought to be a result of the targeting of different parts of the tumour, as described above.

Other agents have also been shown to enhance the activity of DMXAA in xenograft studies. Although the exact mechanism of action of DMXAA is not understood, it is believed to cause upregulation of various cytokines, and compounds with similar activity appear to enhance its effectiveness. These include tumour necrosis factor stimulating compounds and immunomodulatory compounds such as intracellular adhesion molecules (ICAMs).

Diclofenac, an NSAID that has been shown to enhance the anti-tumour activity of DMXAA, is believed to affect the PK of DMXAA via competition for metabolic pathways. At a concentration of 100 μM, diclofenac has been shown to significantly inhibit glucoronidation (>70%) and 6-methylhydroxylation (>54%) of DMXAA in mouse and human liver microsomes. In vivo, diclofenac (100 mg/kg i.p.) has been shown to result in a 24% and 31% increase in the plasma DMXAA AUC (area under the plasma concentration-time curve) and a threefold increase in T_{1/2} (P<0.05) in male and female mice respectively (see Zhou et al. (2001) Cancer Chemother. Pharmacol. 47, 319-326). Other NSAIDs have been shown to have a similar effect.

Similarly to diclofenac, thalidomide, which is approved for erythema nodosum leprosum (ENL), seems to enhance the activity of DMXAA. Thalidomide is also known to have anti-angiogenic effects but the synergy is caused by effect on metabolism of DMXAA. It competes for glucuronidation, prolonging DMXAA's presence at therapeutic levels in tumour tissue. Thalidomide increases the AUC of DMXAA by 1.8 times in plasma, liver and spleen and by three times in tumour (see Kestell et al. (2000) Cancer Chemother. Pharmacol. 46(2), 135-41).

### DESCRIPTION OF THE INVENTION

In a first aspect, the present invention provides a method for modulating neoplastic growth, which comprises administering to a mammal, including a human, in need of treatment an effective amount of formula (I):

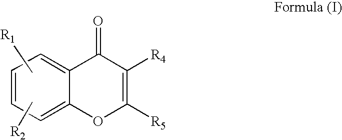

wherein:
(a) R₄ and R₅ together with the carbon atoms to which they are joined, form a 6-membered aromatic ring having a substituent —R₃ and a radical —(B)—COOH where B is a linear or branched substituted or unsubstituted C₁-C₆ alkyl radical, which is saturated or ethylenically unsaturated, and wherein R₁, R₂ and R₃ are each independently selected from the group consisting of H, C₁-C₆ alkyl, halogen, CF₃, CN, NO₂, NH₂, OH, OR, NHCOR, NHSO₂R, SR, SO₂R or NHR, wherein each R is independently C₁-C₆ alkyl optionally substituted with one or more substituents selected from hydroxy, amino and methoxy; or

(b) one of R₄ and R₅ is H or a phenyl radical, and the other of R₄ and R₅ is H or a phenyl radical which may optionally be substituted, thenyl, furyl, naphthyl, a C₁-C₆ alkyl, cycloalkyl, or aralkyl radical; R₁ is H or a C₁-C₆ alkyl or C₁-C₆ alkoxy radical; R₂ is the radical —(B)—COOH where B is a linear or branched substituted or unsubstituted C₁-C₆ alkyl radical, which is saturated or ethylenically unsaturated,

or a pharmaceutically acceptable salt, ester or prodrug thereof and concomitantly or sequentially administering an EGFR signalling pathway inhibitor.

Where (B) in the radical —(B)—COOH is a substituted C₁-C₆ alkylene radical, the substituents may be alkyl, for example methyl, ethyl, propyl or isopropyl, or halide such as fluoro, chloro or bromo groups. In one example the substituent is methyl.

In one embodiment of the first aspect of the invention, the compound of the formula (I) as defined above may be a compound of the formula (II):

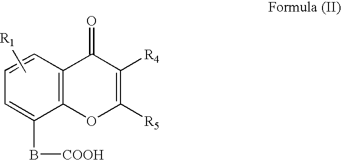

where R₁, R₄, R₅ and B are as defined above for formula (I) in part (b).

In a further embodiment of the first aspect of the invention, the compound of formula (I) as defined above may be a compound of the formula (III):

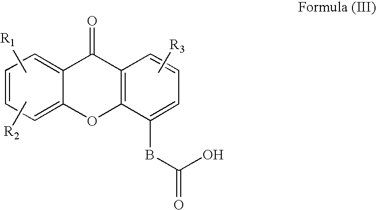

wherein R₁, R₂ and R₃ are each independently selected from the group consisting of H, C₁-C₆ alkyl, halogen, CF₃, CN, NO₂, NH₂, OH, OR, NHCOR, NHSO₂R, SR, SO₂R or NHR, wherein each R is independently C₁-C₆ alkyl optionally substituted with one or more substituents selected from hydroxy, amino and methoxy;
wherein B is as defined for formula (I) above;
and wherein in each of the carbocyclic aromatic rings in formula (I), up to two of the methine (—CH═) groups may be replaced by an aza (—N═) group;
and wherein any two of R₁, R₂ and R₃ may additionally together represent the group —CH═CH—CH═CH—, such that this group, together with the carbon or nitrogen atoms to which it is attached, forms a fused 6-membered aromatic ring.

For example, the compound of formula (III) may be a compound of the formula (IV):

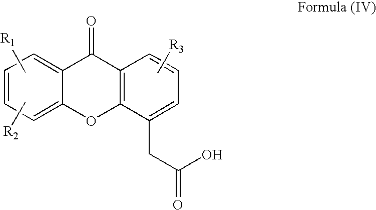

wherein R, R₁, R₂ and R₃ are as defined for formula (III).

In one embodiment of the compound of formula (IV), R₂ is H, one of R₁ and R₃ is selected from the group consisting of C₁-C₆ alkyl, halogen, CF₃, CN, NO₂, NH₂, OH, OR, NHCOR, NHSO₂R, SR, SO₂R or NHR, wherein each R is independently C₁-C₆ alkyl optionally substituted with one or more substituents selected from hydroxy, amino and methoxy, and the other of R₁ and R₃ is H.

For example, the compound of formula (IV) may be of the formula (V):

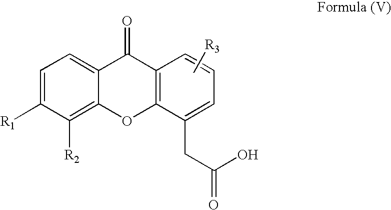

wherein R, R₁, R₂ and R₃ are as defined for formula (IV).

The compound of formula (V) may be, for example, 5,6-dimethylxanthenone-4-acetic acid (DMXAA), or a pharmaceutically acceptable salt, ester or prodrug thereof.

In one embodiment of the invention the EGFR signalling pathway inhibitor is a monoclonal antibody.

In one embodiment of the invention the EGFR signalling pathway inhibitor is Erbitux™ (cetuximab).

In one embodiment of the invention the EGFR signalling pathway inhibitor is a tyrosine kinase inhibitor.

In one embodiment of the invention the EGFR signalling pathway inhibitor is Tarceva™ (erlotinib).

In one embodiment of the invention the EGFR signalling pathway inhibitor is Iressa™ (gefitinib).

In another aspect, the present invention provides the use of a EGFR signalling pathway inhibitor for the manufacture of a medicament (e.g. of a unit dose of a medicament), for simultaneous, separate or sequential administration with the compound of formula (I) as defined above or a pharmaceutically acceptable salt, ester or prodrug thereof (e.g. a unit dose of the compound of formula (I) as defined above or a pharmaceutically acceptable salt, ester or prodrug thereof), for the modulation of neoplastic growth.

In another aspect, the present invention provides the use of the compound of formula (I) as defined above or a pharmaceutically acceptable salt, ester or prodrug thereof for the manufacture of a medicament (e.g. a unit dose of a medicament) for simultaneous, separate or sequential administration with the EGFR signalling pathway inhibitor (e.g. a unit dose of the EGFR signalling pathway inhibitor) for the modulation of neoplastic growth.

According to one aspect, the neoplastic growth is a tumour and/or a cancer.

In a further aspect, the neoplastic growth is one or more of ovarian, prostate, lung, pancreatic, colorectal, and head and neck cancer.

In a further aspect, there is provided a pharmaceutical formulation comprising a combination of the compound of formula (I) as defined above or a pharmaceutically acceptable salt, ester or prodrug thereof (e.g. in a unit dose) and an EGFR signalling pathway inhibitor (e.g. in a unit dose).

In one embodiment there is provided a compound according to formula (I) or a pharmaceutically acceptable salt, ester or prodrug thereof and an EGFR signalling pathway inhibitor for use (in combination) as a medicament for modulation of neoplastic growth.

The invention further provides a process for the preparation of a pharmaceutical formulation which process comprises bringing into association a combination of the compound of formula (I) as defined above or a pharmaceutically acceptable salt, ester or prodrug thereof (e.g. a unit dose of the compound of formula (I) as defined above or a pharmaceutically acceptable salt, ester or prodrug thereof) and an EGFR signalling pathway inhibitor (e.g. a unit dose of the EGFR signalling pathway inhibitor), optionally with one or more pharmaceutically acceptable carriers therefor. For example, the pharmaceutical formulation may be in a unit dose.

Pharmaceutical formulations comprise the active ingredients (that is, the combination of a compound of formula (I) as defined above or pharmaceutically acceptable salt, ester or prodrug thereof and the growth factor inhibitor, for example EGFR signalling pathway inhibitor), for example together with one or more pharmaceutically acceptable carriers therefor and optionally other therapeutic and/or prophylactic ingredients. The carrier(s) must be acceptable in the sense of being compatible with the other ingredients in the formulation and not deleterious to the recipient thereof.

The compound of formula (I) as defined above or a pharmaceutically acceptable salt, ester or prodrug thereof and the EGFR signalling pathway inhibitor may be administered simultaneously, separately or sequentially.

In one embodiment, the pharmaceutically acceptable salt is a sodium salt.

The amount of a combination of a compound of formula (I) as defined above or pharmaceutically acceptable salt, ester or prodrug thereof and an EGFR signalling pathway inhibitor required to be effective as a modulator of neoplastic growth will, of course, vary and is ultimately at the discretion of the medical practitioner. The factors to be considered include the route of administration and nature of the formulation, the mammal's bodyweight, age and general condition and the nature and severity of the disease to be treated.

A suitable effective dose of a compound of formula (I) as defined above, or a pharmaceutically acceptable salt thereof, for administration, simultaneously, separately or sequentially, with an EGFR signalling pathway inhibitor, for the treatment of cancer is in the range of 600 to 4900 mg/m². For example from 2500 to 4000 mg/m², from 1200 to 3500 mg/m², more suitably from 2000 to 3000 mg/m², particularly from 1200 to 2500 mg/m², more particularly from 2500 to 3500 mg/m², preferably from 2250 to 2750 mg/m².

It is of course also possible to base dosages upon the weight of a patient. For example, a dosage of a compound of formula (I) as defined above, or a pharmaceutically acceptable salt thereof, for administration, simultaneously, separately or sequentially, with an EGFR signalling pathway inhibitor, for the treatment of cancer may be in the range of 15 to 125 mg/kg body weight may be administered. More preferably, the dosage is from 30 to 80 mg/kg, or 30 to 70 mg/kg.

In one embodiment the Erbitux™ may be administered in a loading dose of 250 to 500 mg/m² (e.g. about 400 mg/m²) and then weekly doses of 150 to 350 mg/m² (e.g. about 250 mg/m²).

As above, the dosage for Erbitux™ may be based upon the weight of a patient. For example, Erbitux™ may be administered in a loading dose of 6 to 13 mg/kg (e.g. about 10 mg/kg) and then weekly doses of 4 to 9 mg/kg (e.g. about 6 mg/kg).

In one embodiment the Iressa™ and Tarceva™ may be administered in an amount of one 100 to 350 mg tablet daily. For example, Iressa™ may be administered in an amount of one 250 mg tablet daily, and the Tarceva™ may be administered in an amount of one 150 mg tablet daily.

The pharmaceutical formulation may be delivered intravenously (e.g. a formulation containing Erbitux™) or orally (e.g. a formulation containing Iressa™ or Tarceva™). The pharmaceutical composition for intravenous administration may be used in the form of sterile aqueous solutions or in an oleaginous vehicle which may contain other substances, for example, enough salts or glucose to make the solution isotonic with blood. The aqueous solutions may be buffered (e.g. to a pH from 3 to 9), if necessary.

The pharmaceutical formulations (e.g. containing Iressa™ or Tarceva™) may, for example, be administered orally in one or more of the forms of tablets, capsules, ovules, elixirs, solutions or suspensions, which may contain flavouring or colouring agents, for immediate-, delayed-, modified-, sustained-, pulsed- or controlled-release applications.

If the pharmaceutical formulation is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid formulations of a similar type may also be employed as fillers in gelatin capsules. Preferred excipients in this regard include lactose, starch, cellulose, milk sugar or high molecular weight polyethylene glycols. For aqueous suspensions and/or elixirs, the compound may be combined with various sweetening or flavouring agents, colouring matter or dyes, with emulsifying and/or suspending agents and with diluents such as water, ethanol, propylene glycol and glycerin, and combinations thereof.

Pharmaceutical formulations suitable for oral administration may, where appropriate, be conveniently presented in discrete dosage units and may be prepared by any of the methods well known in the art of pharmacy. All methods include the step of bringing into association the active ingredients with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of the active ingredients. A tablet may be made by compression or moulding, optionally with one or more accessory ingredients. Compressed tablets may be prepared by compressing in a suitable machine the active compounds in a free-flowing form such as a powder or granules optionally mixed with a binder, lubricant, inert diluent, lubricating agent, surface-active agent or dispersing agent. Moulded tablets may be made by moulding an inert liquid diluent. Tablets may be optionally coated and, if uncoated, may optionally be scored. Capsules may be prepared by filling the active ingredients, either alone or in admixture with one or more accessory ingredients, into the capsule shells and then sealing them in the usual manner. Cachets are analogous to capsules wherein the active ingredients together with any accessory ingredient(s) are sealed in a rice paper envelope. The compound of formula (I) or a pharmaceutically acceptable salt or ester may also be formulated as dispersible granules, which may for example be suspended in water before administration, or sprinkled on food. The granules may be packaged e.g. in a sachet.

The active ingredients may also be formulated as a solution or suspension for oral administration. Formulations suitable for oral administration wherein the carrier is a liquid may be presented as a solution or a suspension in an aqueous liquid or a non-aqueous liquid, or as an oil-in-water liquid emulsion.

As used herein, the term “prodrug” includes entities that have certain protected group(s) and which may not possess pharmacological activity as such, but may, in certain instances, be administered (such as orally or parenterally) and thereafter metabolised in the body to form the agents which are pharmacologically active.

Furthermore, the invention also provides a kit comprising in combination for simultaneous, separate or sequential use in modulating neoplastic growth, the compound according to formula (I) as defined above or a pharmaceutically acceptable salt, ester or prodrug thereof and an EGFR signalling pathway inhibitor.

### DESCRIPTION OF THE FIGURES

FIG. 1: shows the average tumour volume (relative to the average volume on the first day of treatment) for A549 (lung carcinoma) xenografts observed for an untreated control group of mice and for mice given (i.e. treated with) Erbitux™ (alone), DMXAA (alone), or a combination of Erbitux™ and DMXAA.

FIG. 2: is a representation of the same data used to generate FIG. 1, but expressed in terms of the percentage of mice having tumour volume less than four times the volume measured on the first day of treatment.

### EXAMPLES

### Example 1

### Method

Xenografts for human lung cancer are set-up in groups of nude, athymic mice. The cell line selected was A549 (ATCC number CCL-185), a lung carcinoma.

The A549 was selected as DMXAA has previously been shown to be effective in these cell lines when used in combination with paclitaxel or 5-FU in xenograft studies.

| | | | Dose level | |
|---|---|---|---|---|
| Group | Cell line | Treatment | (mg/kg) | No. of mice |
| 1 | A549 | Untreated control | — | 10 |
| 2 | A549 | DMXAA | 21 | 10 |
| 3 | A549 | Erbitux ™ | 33* | 10 |
| 4 | A549 | Erbitux ™/DMXAA | 33* & 21 | 10 |

*Calculated from dose of 1 mg/mouse.

For this study, DMXAA is given twice in each of Weeks 1 and 4 of the study. Erbitux™ is given twice weekly for four weeks.

Xenografts are measured two or three times per week and their absolute volume recorded; xenograft tumour volume relative to that recorded on Day 0 (V₀) is then calculated. The time taken to reach a relative tumour volume of 3×V₀ is used as a surrogate marker for survival.

### Results

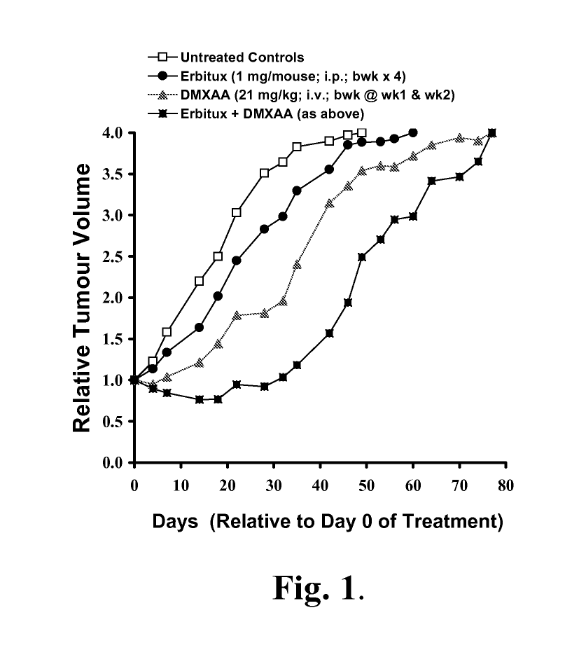

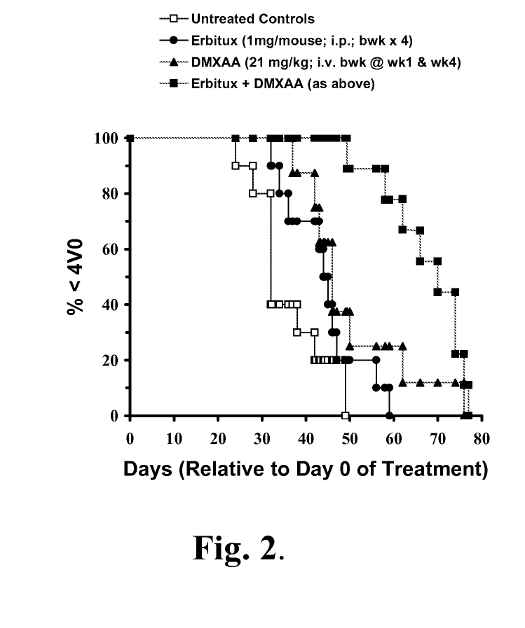

Tables 1 and 2 below, as well as FIGS. 1 and 2 show that the combination of Erbitux™ and DMXAA provides an unexpected synergistic effect in delaying tumour growth.

TABLE 1
Results of studies with A549 xenografts.

| | Dose | | | | Regression | |
|---|---|---|---|---|---|---|
| | (mg/kg by | Drug | Median VQT | Tumour Growth | Duration^{b} | TTP^{c} |
| Group | injection) | deaths | (Range; days) | Delay^{a} (Days) | (Days) | (Days) |
| Erbitux ™ | 33^{d} | 0/10 | 44 | 12 | 0 | 4 |
| DMXAA | 21 | 2/10 | 48 | 16 | 0 | 16 |
| Erbitux ™/ | 33^{d} + 21 | 1/10 | 70 | 38 | 28 | 34 |
| DMXAA | | | | | | |

^{a}The difference in days for treated versus control tumours to quadruple in volume (control tumours quadrupled in 17 (14-23) days).
^{b}Tumour regression duration is the number of days that the tumour volume is less than the original treatment volume.
^{c}TTP: Median time to disease progression.
^{d}Calculated from dose of 1 mg/mouse.

TABLE 2
Results of studies with A549 xenografts.

| Dose | |
|---|---|
| (mg/kg by | Response^{e} |

| Group | injection) | PD | PR | SD | CR |
|---|---|---|---|---|---|
| Erbitux ™ | 33^{d} | 0/10 | 44 | 12 | 0 |
| DMXAA | 21 | 2/10 | 48 | 16 | 0 |
| Erbitux ™/ | 33^{d} + 21 | 1/10 | 70 | 38 | 28 |
| DMXAA | | | | | |

^{d}Calculated from dose of 1 mg/mouse.
^{e}PD: Progressive Disease (≧50% increase in tumour size)
PR: Partial Response (≧50% reduction in tumour size sustained over two weeks)
SD: Stable Disease (does not satisfy criteria for PR or PD)
CR: Complete Response (cure; undetectable tumour over two weeks)

### ABBREVIATIONS

AUC=area under curve (plasma concentration vs. time)

### CR=Complete Response

DMXAA=5,6-dimethylxanthenoneacetic acid
EGF=endothelial growth factor
EGFR=endothelial growth factor receptor
ENL=erythema nodosum leprosum
5-FU=5-fluorouracil
HPC=hydroxypropylcellulose
HPMC=hydroxymethylcellulose
ICAM=intracellular adhesion molecule
i.p.=intraperitoneal
MRI=magnetic resonance imaging

### PD=Progressive Disease

PK=pharmacokinetics

### PR=Partial Response

### SD=Stable Disease

TTP=median time to disease progression
VDA=vascular disrupting agent
VQT=(tumour) volume quadrupling time

1. A method for modulating neoplastic growth, which comprises administering to a mammal, including a human, in need of treatment an effective amount of a compound of Formula (I): 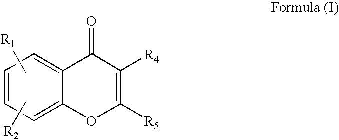
wherein:
(a) R₄ and R₅ together with the carbon atoms to which they are joined, form a 6-membered aromatic ring having a substituent —R₃ and a radical —(B)—COOH where B is a linear or branched substituted or unsubstituted C₁-C₆ alkyl radical, which is saturated or ethylenically unsaturated, and wherein R₁, R₂ and R₃ are each independently selected from the group consisting of H, C₁-C₆ alkyl, halogen, CF₃, CN, NO₂, NH₂, OH, OR, NHCOR, NHSO₂R, SR, SO₂R or NHR, wherein each R is independently C₁-C₆ alkyl optionally substituted with one or more substituents selected from hydroxy, amino and methoxy; or
(b) one of R₄ and R₅ is H or a phenyl radical, and the other of R₄ and R₅ is H or a phenyl radical which may optionally be substituted, thenyl, furyl, naphthyl, a C₁-C₆ alkyl, cycloalkyl, or aralkyl radical; R1 is H or a C₁-C₆ alkyl or C₁-C₆ alkoxy radical; R₂ is the radical —(B)-000H where B is a linear or branched substituted or unsubstituted C₁-C₆ alkyl radical, which is saturated or ethylenically unsaturated,
or a pharmaceutically acceptable salt, ester or prodrug thereof and concomitantly or sequentially administering an EGFR signalling pathway inhibitor.

2. The method according to claim 1 wherein the compound of Formula (I) is a compound of Formula (II): 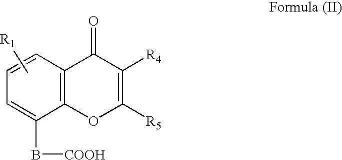
wherein R₁, R₄, R₅ and B are as defined for formula (I) in claim 1 part (b).

3. The method according to claim 1 wherein the compound of Formula (I) is a compound of Formula (III): 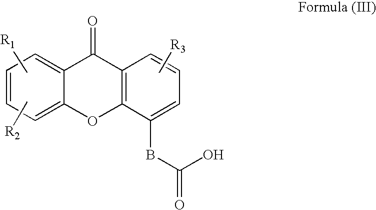
wherein R₁, R₂ and R₃ are each independently selected from the group consisting of H, C₁-C₆ alkyl, halogen, CF₃, CN, NO₂, NH₂, OH, OR, NHCOR, NHSO₂R, SR, SO₂R or NHR, wherein each R is independently C₁-C₆ alkyl optionally substituted with one or more substituents selected from hydroxy, amino and methoxy;
wherein B is as defined for formula (I) in claim 1;
and wherein in each of the carbocyclic aromatic rings in formula (I), up to two of the methine (—CH═) groups may be replaced by an aza (—N═) group;
and wherein any two of R₁, R₂ and R₃ may additionally together represent the group —CH═CH—CH═CH—, such that this group, together with the carbon or nitrogen atoms to which it is attached, forms a fused 6 membered aromatic ring.

4. The method according to claim 3, wherein the compound of Formula (III) is a compound of Formula (IV): 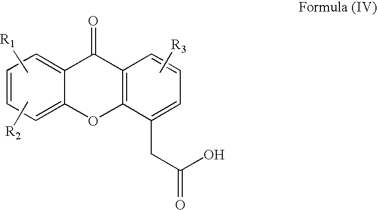
wherein R, R₁, R₂ and R₃ are as defined for formula (III) claim 3.

5. The method according to claim 4 wherein the compound of Formula (IV) is a compound of Formula (V): 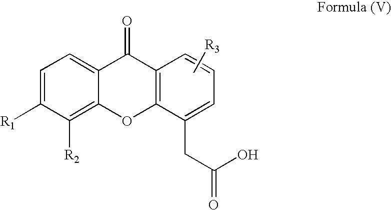
wherein R, R₁, R₂ and R₃ are as defined for formula (IV) in claim 4.

6. The method according to claim 1, wherein the compound of Formula (I) is DMXAA or a pharmaceutically acceptable salt, ester or prodrug thereof.

7. A method according to claim 1 wherein the compound of formula (I) or a pharmaceutically acceptable salt, ester or prodrug thereof and the EGFR signalling pathway inhibitor are administered concomitantly.

8. A method according to claim 1 wherein the compound of formula (I) or pharmaceutically acceptable salt, ester or prodrug thereof and the EGFR signalling pathway inhibitor are administered sequentially.

9. (canceled)

10. (canceled)

11. (canceled)

12. A pharmaceutical formulation comprising a combination of a compound of formula (I), (II), (III), (IV) or (V), as defined in any one of claims 1 to 6, or a pharmaceutically acceptable salt, ester or prodrug thereof and an EGFR signalling pathway inhibitor.

13. A pharmaceutical formulation according to claim 12 wherein the formulation is adapted for intravenous or oral administration.

14. A pharmaceutical formulation according to claim 12 or 13 which additionally comprises a pharmaceutically acceptable carrier.

15. A kit comprising in combination for simultaneous, separate or sequential use in modulating neoplastic growth, a compound a compound of formula (I), (II), (III), (IV) or (V), as defined in any one of claims 1 to 6, or a pharmaceutically acceptable salt, ester or prodrug thereof and an EGFR signalling pathway inhibitor.

16. The method of claim 1, wherein the EGFR signalling pathway inhibitor is a monoclonal antibody.

17. The method of claim 1, wherein the EGFR signalling pathway inhibitor is cetuximab.

18. The method of claim 1, wherein the EGFR signalling pathway inhibitor is a tyrosine kinase inhibitor.

19. The method of claim 1, wherein the EGFR signalling pathway inhibitor is erlotinib or gefitinib.

20. (canceled)

21. The pharmaceutical formulation of claim 12, wherein the EGFR signalling pathway inhibitor is a monoclonal antibody.

22. The pharmaceutical formulation of claim 12, wherein the EGFR signalling pathway inhibitor is cetuximab.

23. The pharmaceutical formulation of claim 12, wherein the EGFR signalling pathway inhibitor is a tyrosine kinase inhibitor.

24. The pharmaceutical formulation of claim 12, wherein the EGFR signalling pathway inhibitor is erlotinib or gefitinib.

25. The kit of claim 15, wherein the EGFR signalling pathway inhibitor is a monoclonal antibody.

26. The kit of claim 15, wherein the EGFR signalling pathway inhibitor is cetuximab.

27. The kit of claim 15, wherein the EGFR signalling pathway inhibitor is a tyrosine kinase inhibitor.

28. The kit of claim 15, wherein the EGFR signalling pathway inhibitor is erlotinib or gefitinib.

29. A pharmaceutical formulation according to claims 13 which additionally comprises a pharmaceutically acceptable carrier.
